(19) United States
(12) Patent Application Publication
(10) Pub. No.: US 2009/0318728 A1
(43) Pub. Date: Dec. 24, 2009

(54) PROCESSES FOR PREPARING PRODRUGS OF GABAPENTIN AND INTERMEDIATES THEREOF

(75) Inventors: Elena Ben Moha-Lerman, Kiryat Ono (IL); Valerie Niddam-Hildesheim, Kadima (IL)

Correspondence Address: MERCHANT & GOULD PC, P.O. BOX 2903, MINNEAPOLIS, MN, 55402-0903, US

(73) Assignee: TEVA PHARMACEUTICAL INDUSTRIES LTD., Petah-Teqva (IL)

(21) Appl. No.: 12/490,936

(22) Filed: Jun. 24, 2009

Related U.S. Application Data
(60) Provisional application No. 61/133,097, filed on Jun. 24, 2008.
(60) Provisional application No. 61/134,652, filed on Jul. 10, 2008.
(60) Provisional application No. 61/192,970, filed on Sep. 22, 2008.

(51) Int. Cl. C07C 329/00; C07C 229/00; C07C 69/34

(52) U.S. Cl. 558/248; 560/125; 560/190

(57) ABSTRACT

Gabapentin prodrugs and intermediates thereof are described.

### CROSS-REFERENCE TO RELATED APPLICATIONS

This application claims the benefit of U.S. Provisional Patent Application Ser. No. 61/133,097, filed Jun. 24, 2008, 61/134,652, filed Jul. 10, 2008, and 61/192,970, filed Sep. 22, 2008, each of which is incorporated herein by reference.

### FIELD OF THE INVENTION

The present invention relates to processes for preparing intermediates of gabapentin enacarbil, processes for preparing gabapentin enacarbil derivatives, and processes for preparing prodrugs of Gabapentin, as well as the preparation of gabapentin enacarbil.

### BACKGROUND OF THE INVENTION

Gabapentin (“GBP”), 1-(aminomethyl)cyclohexaneacetic acid is described according to the following formula:

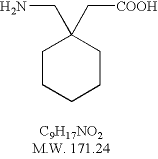

GBP is a white to off-white crystalline solid with a pKa1 of 3.7 and a pKa2 of 10.7. GBP is marketed by Pfizer under the trade name Neurontin®.

GBP is used in the treatment of cerebral diseases such as epilepsy. In animal models of analgesia, GBP prevents allodynia (pain-related behavior in response to a normally innocuous stimulus) and hyperalgesia (exaggerated response to painful stimuli). GBP also decreases pain related responses after peripheral inflammation. Animal test systems designed to detect anticonvulsant activity, proved that GBP prevents seizures as do other marketed anticonvulsants.

Gabapentin Enacarbil (“GBPE”), 1-{[(α-isobutanoyloxyethoxy)carbonyl]-aminomethyl}-1-cyclohexane acetic acid, is a transported prodrug of GBP and is described according to the following formula:

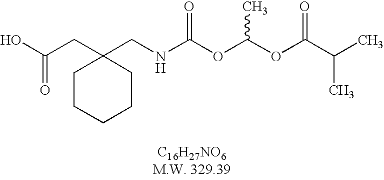

GBPE is designed to improve some of the bioavailability limitations that are known in GBP. GBPE is designed to be recognized by high-capacity transport proteins expressed all along the intestinal tract, making it suitable for sustained-release formulation for colonic absorption. After absorption, GBPE is rapidly converted to GBP.

GBPE and processes for its preparation are described in U.S. Pat. Nos. 6,818,787, 7,232,924, and 7,227,028.

According to U.S. Pat. No. 7,227,028 (“the US '028 patent”), GBPE is obtained according to a process consisting of 4 steps using a thiol having a C₁-C₄ alkyl. The process described in the US '028 patent is provided below in scheme 1:

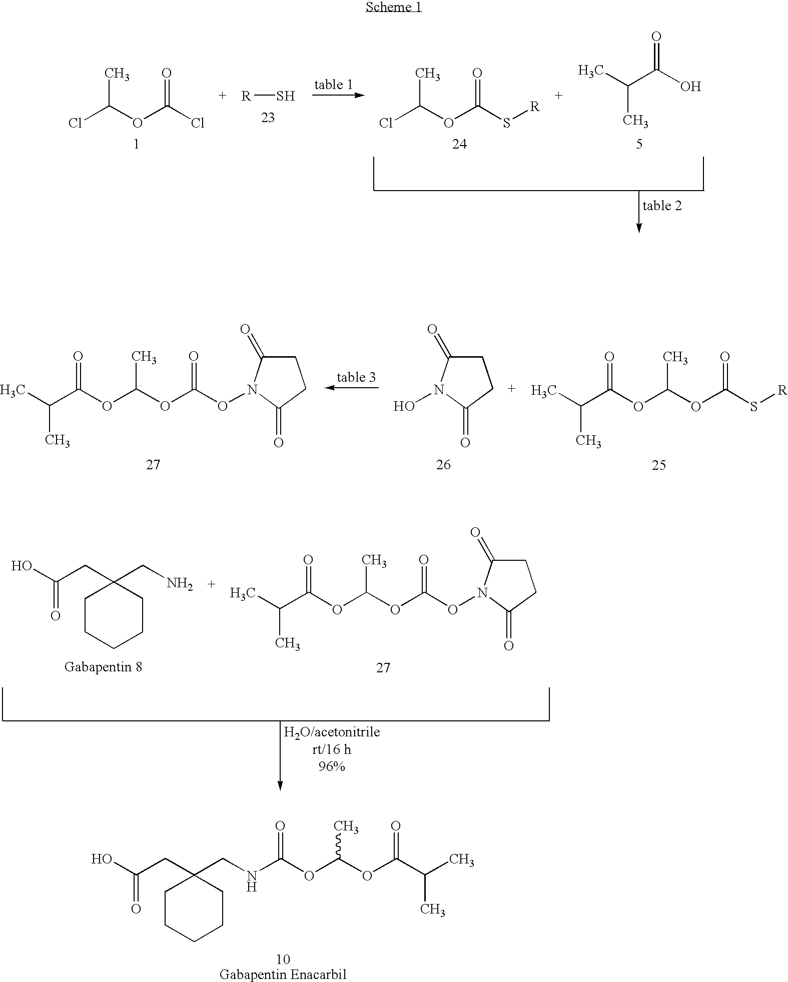

There is a need in the art for improved processes for preparing prodrugs of GBP.

### SUMMARY OF THE INVENTION

In one embodiment, the present invention encompasses chloroethyl carbonate-thiol (“CEC-Thiol”) having the following formula:

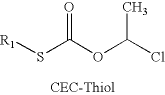

wherein R₁ is C₅-C₂₀ alkyl.

In yet another embodiment, the present invention encompasses acid ethyl carbonate-thiol (“AEC-Thiol”) having the following formula:

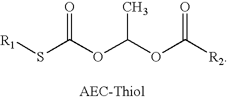

wherein R₁ is C₅-C₂₀ alkyl. In addition, R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms.

In another embodiment, the present invention encompasses a process for the preparation of CEC-Thiol having the following formula:

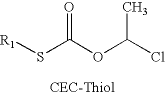

comprising: combining chloroethyl chloroformate (“CEC”) with R₄CH₂SH having the following structure:

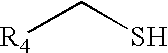

or its salt, wherein R₁ is C₅-C₂₀ alkyl. R₁ is CH₂R₄ and R₄ is C₄-C₁₉ alkyl.

In one embodiment, the present invention encompasses process for preparing a prodrug of GBP comprising converting CEC-thiol, defined above, into a prodrug of GBP.

In another embodiment, the present invention encompasses a process for preparing a prodrug of GBP, comprising: obtaining CEC-thiol according to the process described herein and further converting it to a prodrug of GBP.

In another embodiment, the present invention encompasses a process for the preparation of AEC-Thiol having the following formula:

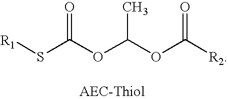

comprising: combining CEC-Thiol of the following formula:

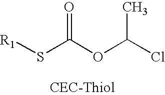

with a carboxylic acid having the formula R₂CO₂H, or its salt, wherein R₁ is C₅-C₂₀ alkyl, and R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms.

In one embodiment, the present invention encompasses a process for preparing a prodrug of GBP, comprising: converting AEC-thiol into a prodrug of GBP.

In one embodiment, the present invention encompasses acid ethyl carbonate-chloride (“AEC-Cl”) of the following formula:

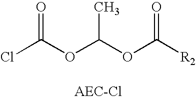

wherein R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms.

In another embodiment, the present invention encompasses a process for preparing AEC-Cl comprising: combining AEC-thiol with a chlorinating agent.

In one embodiment, the present invention encompasses a process for preparing a prodrug of GBP, preferably, GBPE, comprising: combining the intermediate AEC-Cl with a GBP derivative.

In another embodiment, the present invention encompasses a process for preparing a prodrug of GBP, comprising: combining the intermediate AEC-Cl with a GBP derivative in the presence of a catalyst. Typically, the process comprises: combining AEC-Cl with a GBP derivative of the following formula:

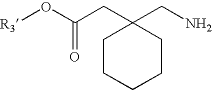

in the presence of a solvent, wherein R₃′ is H, primary ammonium, secondary ammonium, tertiary ammonium, quaternary ammonium or tri-substituted silyl, wherein each individual substituting group of the ammonium or silyl is a hydrogen or a C₁-C₁₀ alkyl, independently of each other.

In yet another embodiment, the present invention encompasses a one-pot process for preparing a prodrug of GBP comprising: combining AEC-thiol with a chlorinating agent; and adding a GBP derivative of the following formula:

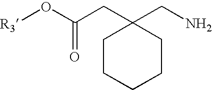

wherein the process is done in the presence of a solvent, wherein R₃′ is H, primary ammonium, secondary ammonium, tertiary ammonium, quaternary ammonium or tri-substituted silyl, wherein each individual substituting group of the ammonium or silyl is a hydrogen or a C₁-C₁₀ alkyl, independently of each other.

In one embodiment, the present invention encompasses a process for preparing a prodrug of GBP, preferably, GBPE comprising: combining the intermediate AEC-Thiol with a GBP derivative in the presence of an activating agent. Preferably, the process comprises: combining the intermediate AEC-Thiol of the following formula:

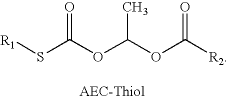

with a GBP derivative of the following formula

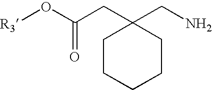

in the presence of an activating agent, wherein R₁ is C₁-C₂₀ alkyl, R₂ is H, C₁-C₁₉ alkyl or C₆-C₁₉ aryl, and R₃′ is H, primary ammonium, secondary ammonium, tertiary ammonium, quaternary ammonium or tri-substituted silyl, wherein each individual substituting group of the ammonium or silyl is a hydrogen or a C₁-C₁₀ alkyl, independently of each other.

### DETAILED DESCRIPTION OF THE INVENTION

As used herein, the term “room temperature” or “RT” refers to a temperature of about 15° C. to about 30° C., preferably, to a temperature of about 20° C. to about 25° C.

As used herein, the term “volume” or “vol” refers to a ml to gram ratio.

As used herein, unless otherwise stated, the term “alkyl” refers to linear, branched, or cyclic hydrocarbon chain radical consisting of carbon and hydrogen atoms, containing no un-saturation, and which is attached to the rest of the molecule by a single bond, e.g., methyl, ethyl, n-propyl, 1-methylethyl(isopropyl), n-butyl, n-pentyl, 1,1-dimethylethyl(t-butyl), and the like.

As used herein, the term “Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

As used herein, the term “gabapentin (“GBP”) prodrug” refers to the product having the following formula:

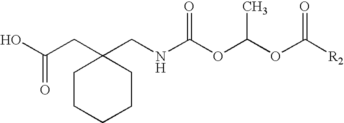

wherein R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms. Preferably, R₂ is isopropyl. When R₂ is isopropyl, the GBP prodrug is gabapentin enacarbil.

As used herein, the term “gabapentin enacarbil (“GBPE”) derivative” refers to the product having the following formula:

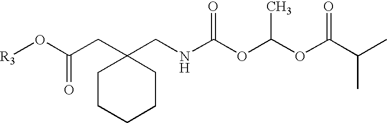

wherein R₃ is C₁-C₁₉ alkyl, allyl, benzyl or tri-substituted silyl, wherein each individual substituting group on the silyl is a hydrogen or a C₁-C₁₀ alkyl, independently of each other, provided that R₃ does not contain more than 19 total carbon atoms. Preferably, R₃ is C₁-C₁₀ alkyl. Preferably, R₃ is tri-methyl silyl, tri-ethyl silyl or tert-butyl dimethyl silyl.

As used herein, the term “GBPE” refers to Gabapentin enacarbil.

As used herein, the term “Activating agent” refers to a material added to a second material or mixture so that a physical or chemical change of the second material or mixture will take place more rapidly or completely. For example, use of an activating agent in the synthesis of a GBP prodrug or a GBPE derivative, as described herein, makes the reaction go faster and/or more completely. Preferred activating agents of the invention include mercury or silver salts of trifluoroacetic or trifluorosulfonic acids.

The present invention provides an efficient way for preparing Gabapentin (“GBP”) prodrugs, specifically GBPE, and derivatives of GBPE in terms of yield and/or number of synthetic steps.

The processes provided in the present invention are depicted below in scheme 2, wherein two synthetic pathways (A and B) are presented for preparing GBP prodrugs, GBPE and GBPE derivatives.

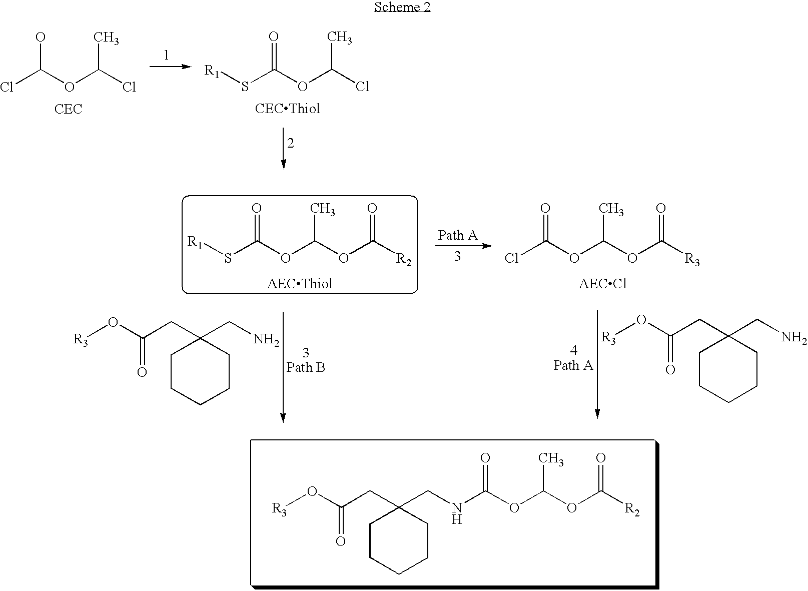

In one embodiment, the present invention encompasses chloroethyl carbonate-thiol (“CEC-Thiol”) having the following formula:

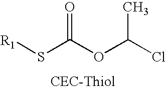

wherein R₁ is C₅-C₂₀ alkyl. Preferably, R₁ is C₅-C₁₈ alkyl, more preferably, C₁₀-C₁₅ alkyl, most preferably, linear C₁₂ alkyl.

In another embodiment, the present invention encompasses acid ethyl carbonate-thiol (“AEC-Thiol”) having the following formula:

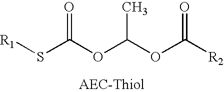

wherein R₁ is C₅-C₂₀ alkyl and R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms. Preferably, R₁ is C₅-C₁₈ alkyl, more preferably, C₁₀-C₁₅ alkyl, most preferably, linear C₁₂ alkyl. Preferably, R₂ is isopropyl.

The longer R₁ is, the smell accompanying AEC-thiol is more bearable, making it easier to handle during production.

In yet another embodiment, the present invention encompasses a process for the preparation of CEC-Thiol having the following formula:

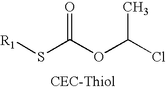

comprising: combining chloroethyl chloroformate (“CEC”) with R₄CH₂SH having the following structure:

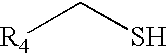

or its salt, wherein R₁ is CH₂R₄, and R₄ is C₄-C₁₉ alkyl. Preferably, the R₄CH₂SH salt is alkaline metal salt, more preferably, potassium or sodium salt (i.e. R₄CH₂SK or R₄CH₂SNa), most preferably, sodium salt. Preferably, R₄ is linear C₁₁ alkyl i.e., the R₄CH₂SH is dodecane thiol.

Preferably, the R₄CH₂SH salt is added with water.

Preferably, a polar non-nucleophilic solvent is further added. Preferably, the polar non-nucleophilic solvent is selected from a group consisting of dimethyl sulfoxide (“DMSO”), acetonitrile (“ACN”), C₂-C₆ ethers, C₃-C₆ ketones, dichloroethane, dichloromethane (“DCM”), chloroform, toluene and mixtures thereof More preferably, the solvent is DCM.

Preferably, after the addition of the solvent, a reaction mixture is obtained.

Preferably, when the R₄CH₂SH is not added in its salt form, an organic or inorganic base is added to the reaction mixture. Preferably, the organic base is a tertiary amine base. More preferably, the base is N-methylmorpholine. Preferably, the base is added dropwise.

Preferably, when R₄CH₂SH is added as a salt, a phase transfer catalyst is added to the reaction mixture. Appropriate phase transfer catalysts are known to those of ordinary skill in the art. Preferably, the phase transfer catalyst is tetra-n-butylammonium bromide (“TBAB”).

Optionally, the reaction mixture is maintained while stirring to obtain CEC-Thiol. Preferably, the stirring is for about 6 hours to about 48 hours. More preferably, the stirring is for about 16 hours. Preferably, the stirring is done at about room temperature.

Optionally, prior to the maintaining step, the reaction mixture is cooled. Preferably, the cooling is done to a temperature of about 15° C. to about 0° C. More preferably, the cooling is done to a temperature of about 0° C.

Preferably, the process comprises dissolving CEC in a polar non-nucleophilic solvent; optionally adding a phase transfer catalyst; adding R₄CH₂SH or its salt; and isolating the product.

The CEC-thiol may be isolated by any method known in the art. Preferably, the isolation is done by diluting the reaction mixture with CH₂Cl₂, washing with water, saturated NaHCO₃ and brine. In the isolation step two phases are obtained which are further separated. The organic phase may be dried over Na₂SO₄ and the solvent is removed, preferably by distillation to give the desired product. Optionally, filtration is also performed.

The present invention encompasses process for preparing a prodrug of GBP comprising converting CEC-thiol, defined above, into a prodrug of GBP, preferably, to GBPE.

In one embodiment, the present invention encompasses a process for preparing a prodrug of GBP comprising: obtaining CEC-thiol according to the process described above and further converting it to a prodrug of GBP, preferably, to GBPE.

In one embodiment, the present invention encompasses a process for the preparation of AEC-Thiol having the following formula:

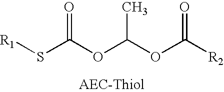

comprising: combining CEC-Thiol of the following formula:

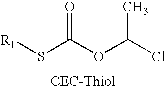

with a carboxylic acid having the formula R₂CO₂H, or its salt, wherein R₁ is C₅-C₂₀ alkyl, and R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms. Preferably, R₂ is isopropyl.

Preferably, when the carboxylic acid is not added as a salt, an organic or inorganic base is further added with a carboxylic acid. Preferably, the organic base is a secondary or tertiary amine base. More preferably, the organic base is diisopropylethylamine. The carboxylic acid added in the solution with the base is the same as the carboxylic acid used previously in the process.

After combining the CEC-Thiol, the carboxylic acid and the solution of the base with the carboxylic acid, a reaction mixture is obtained. Typically, the reaction mixture is obtained by adding a solution of CEC-thiol and carboxylic acid to a solution of an organic or inorganic base and a carboxylic acid. Preferably, the reaction mixture is heated to a temperature of about 40° C. to about 60° C. More preferably, the reaction is heated to a temperature of about 55° C. Preferably, the heating is done for about 6 hours to about 48 hours. More preferably, the heating is done for about 16 hours.

The AEC-thiol may be recovered by any method known in the art. Preferably, the recovery is done by adding a water immiscible polar organic solvent and water to obtain a two phase system; separating the phases; washing the organic phase with NaHCO₃ and brine; and removing the solvent to obtain the final product. The solvent removal may be done by either drying over Na₂SO₄, filtration, distillation or combinations thereof Preferably, the water immiscible polar organic solvent is ether or EtOAc. The obtained product may be further purified by column chromatography.

Preferably, AEC-thiol is obtained in a yield of more than about 95%. More preferably, AEC-thiol is obtained in a yield of more than about 95.5%.

The present invention provides a process for preparing a prodrug of GBP comprising: converting AEC-thiol into a prodrug of GBP, preferably, into GBPE.

In one embodiment, the present invention provides a process for preparing a prodrug of GBP comprising: obtaining AEC-Thiol according to the process described above and further converting it to a prodrug of GBP, preferably, into GBPE.

In another embodiment, the present invention encompasses acid ethyl carbonate-chloride (“AEC-Cl”), having the following formula

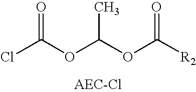

wherein R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms. Preferably, R₂ is isopropyl.

In another embodiment, the present invention encompasses a process for preparing AEC-Cl comprising: combining AEC-thiol, defined above, with a chlorinating agent. Preferably, the chlorinating agent is selected from a group consisting of: SO₂Cl₂, SOCl₂, POCl₃, PCl₃ and PCl₅. More preferably the chlorinating agent is SO₂Cl₂ (“sulfuryl chloride”).

Preferably, AEC-thiol is added cooled, preferably at a temperature of about 5° C. to about 0° C., more preferably, at a temperature of about 0° C.

The chlorinating agent is added to the cooled AEC-thiol to obtain a reaction mixture which may be further maintained while stirring. Preferably, the stirring is for about 5 minutes to about 45 minutes. More preferably, the stirring is for about 30 minutes. The stirring may be further performed at about room temperature, preferably for about 30 minutes to about 120 minutes, more preferably, for about 90 minutes.

Optionally, a solvent is further added to the reaction mixture. Preferably, the solvent added is an inert polar solvent. More preferably, the solvent is selected from a group consisting of C₅ to C₁₂ hydrocarbons, C₆ to C₁₂ aromatic hydrocarbons, and C₁ to C₂ chloroalkanes. Most preferably, the solvent is selected from a group consisting of toluene, CH₂Cl₂, dichloroethane and chloroform.

Optionally, the process is done in the presence of a catalyst. Suitable catalysts would be apparent to those of ordinary skill in the art. Preferably, the catalyst is Lewis acid or irradiation. Preferably, the Lewis acid is AlCl₃ or FeCl₂.

The present invention encompasses a process for preparing a prodrug of GBP, comprising converting AEC-Cl into a prodrug of GBP.

In one embodiment, the present invention encompasses a process for preparing a prodrug of GBP, preferably, GBPE comprising: combining the intermediate AEC-Cl with a GBP derivative of the following formula:

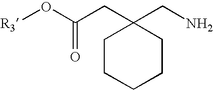

in the presence of a solvent, wherein R₃′ is H, primary ammonium, secondary ammonium, tertiary ammonium, quaternary ammonium or tri-substituted silyl, wherein each individual substituting group on the ammonium or the silyl is a hydrogen or a C₁-C₁₀ alkyl, independently of each other, provided that the total number of carbon atoms of R₃′ does not exceed 19. GBP prodrug is as defined above.

In another embodiment, the present invention encompasses a process for preparing GBPE derivative comprising: combining the intermediate AEC-Cl with a GBP derivative of the following formula:

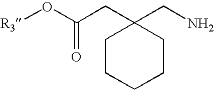

in the presence of a solvent, wherein R₃″ is C₁-C₁₉ alkyl, allyl, or benzyl. Preferably, the obtained GBPE derivative is further converted to GBPE.

Preferably, the solvent added is an inert polar solvent. More preferably, the solvent is selected from a group consisting of C₅ to C₁₂ hydrocarbons, C₆ to C₁₂ aromatic hydrocarbons, C₁ to C₂ chloroalkanes, C₄ to C₁₀ ethers, and combinations of these solvents among themselves or with water. Most preferably, the solvent is toluene, CH₂Cl₂, dichloroethane, methyl tert butyl ether (“MTBE”)/water, or chloroform.

Preferably, the process is done in the presence of a catalyst. Suitable catalysts would be apparent to those of ordinary skill in the art. Preferably, the catalyst is selected from a group consisting of 4-dimethylaminopyridine (“DMAP”), tetra butyl ammonium bromide (“TBAB”) and KI.

Optionally, a base or excess amount of GBP derivative is added. Preferably, the base is an inorganic base or a tertiary amine. More preferably, the base is NaOH. Preferably, the base is added in a solution with MTBE and water.

The obtained product may then be isolated. The isolation may be done by adding water to obtain a two phase system; separating the phases; and removing the solvent from the organic phase. The solvent removal may be done by evaporation. The aqueous phase may be further acidified and extracted. The acidification may be done with NaHSO₄. The extraction may be done with MTBE.

The process for preparing a prodrug of GBP or GBPE derivative from AEC-Thiol can be either performed in two steps or as a one-pot process via in situ formation of AEC-Cl. The two steps process starts from the intermediate AEC-Thiol which is reacted with a chlorinating agent to provide the intermediate AEC-Cl, as described above, and further reacted with GBP derivative, as described above. The one pot process comprises: combining AEC-thiol with a chlorinating agent; and adding a GBP derivative as described above, wherein the process is done in the presence of a solvent. The solvent may be added in the combining step of AEC-thiol with a chlorinating agent, or together with GBP derivative. Preferably, the solvent added is an inert polar solvent. More preferably, the solvent is selected from a group consisting of C₅ to C₁₂ hydrocarbons, C₆ to C₁₂ aromatic hydrocarbons, C₁ to C₂ chloroalkanes, C₄ to C₁₀ ethers, and combinations of these solvents among themselves or with water. Most preferably, the solvent is toluene, CH₂Cl₂, dichloroethane, MTBE/water, or chloroform. The rest of the parameters in the one pot processes are as described above.

In another embodiment, the present invention encompasses a process for preparing a prodrug of GBP, preferably, GBPE comprising: combining the intermediate AEC-Thiol of the following formula:

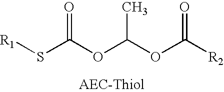

with a GBP derivative of the following formula:

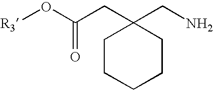

preferably in the presence of an activating agent, wherein R₁ is C₅-C₂₀ alkyl, R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms, and R₃′ is H, primary ammonium, secondary ammonium, tertiary ammonium, quaternary ammonium or tri-substituted silyl, wherein each individual substituting group of the ammonium or silyl is a hydrogen or a C₁-C₁₀ alkyl, independently of each other.

A process for preparing GBPE derivative comprising: combining the intermediate AEC-Thiol of the following formula:

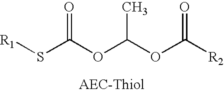

with a GBP derivative of the following formula

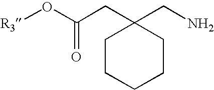

preferably in the presence of an activating agent, wherein R₁ is C₅-C₂₀ alkyl, R₂ is H, C₁-C₁₉ alkyl, or C₆-C₁₉ aryl optionally substituted with one or more C₁-C₁₃ alkyl groups, wherein the alkyl groups are independently selected, provided that R₂ does not contain more than a total of 19 carbon atoms, and R₃″ is C₁-C₁₉, allyl or benzyl.

Preferably, prior to the combining step, AEC-thiol is dissolved in a solvent selected from a group consisting of toluene, tetrahydrofuran (“THF”), MeCN, CH₂Cl₂, and H₂O. Most preferably, the solvent is CH₂Cl₂. The obtained solution is then combined with GBP derivatives in the presence of an activating agent.

Preferably, the activating agent is selected from a group consisting of CF₃CO₂Ag, (CF₃CO₂)₂Hg, CF₃SO₃Ag, and (CF₃SO₃)₂Hg. More preferably, the activating agent is CF₃CO₂Ag.

Optionally, a base or excess amount of GBP derivative is used in the above process. Preferably, the base is an inorganic base or a tertiary amine. More preferably, the base is triethyl amine (“TEA”).

The process may be done for a period of about 2 to about 4 days.

The process may be done at a temperature of about room temperature to about 60° C., preferably, at about 50° C.

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to, limit its scope in any way. Absent statement to the contrary, any combination of the specific embodiments described above are consistent with and encompassed by the present invention.

### EXAMPLES

### Example 1

### Preparation of O-1-chloroethyl S-dodecyl carbonothioate (“CEC dodecane thiol”) (Step 1)

A solution of dodecane thiol (1 eq) and chloroethyl chloroformate (1 eq) in CH₂Cl₂ (5.5 vol) was cooled to 0° C. in an ice-water bath. N-methylmorpholine (1.1 eq) was added dropwise and the reaction mixture was stirred at RT for 16 h. The reaction mixture was diluted with CH₂Cl₂ and washed with water, saturated NaHCO₃ and brine. The organic phase was dried over Na₂SO₄, filtered and concentrated to provide the desired product as colorless liquid.

The above example was repeated several times and the obtained yield was 95% to 100%.

### Example 2

### Preparation of O-1-chloroethyl S-ethyl carbonothioate (“CEC ethyl thiol”) (Step 1)

Chloroethyl-chloroformate (15.7 g; 0.11 mol) was dissolved in CH₂Cl₂ (50 ml) followed by addition of tetrabutylammonium bromide (“TBAB”) catalyst (1 g; 10% wt). Solution of NaSEt (9.2 g; 0.11 mol) in water (60 ml) was added dropwise to the reaction flask and the obtained biphasic mixture was stirred at room temperature for over night. Then the reaction was stopped and the phases were separated. The organic phase was washed with water, dried over Na₂SO₄ and the solvent was removed by distillation to give the desired product as a yellow oil in 60% yield.

### Example 3

### Preparation of S-dodecyl O-1-(isobutyryloxy)ethyl carbonothioate (“AEC-dodecane-thiol”) (Step 2)

O-1-chloroethyl S-dodecyl carbonothioate (1 eq) was dissolved in isobutyric acid (1.5 eq) and the solution was slowly added to a pre-mixed solution of this acid (1.5 eq) and diisopropylethylamine (1.5 eq). The reaction was heated to 55° C. for 16 h and then diluted with ether and washed with water, saturated NaHCO₃ and brine. The organic phase was dried over Na₂SO₄, filtered and concentrated to provide the crude which was purified by distillation or by column chromatography. (Yield: 95.5%)

### Example 4

### Preparation of S-ethyl O-1-(isobutyryloxy)ethyl carbonothioatel (“AEC-ethyl-thiol”) (Step 2)

CEC-Et-thiol (11.11 g; 66 mmol) was dissolved in isobutyric acid (18 ml; 0.198 mol) followed by addition of premixed solution of isobutyric acid (18 ml; 0.198 mol) and diisopropylethylamine (“DIPEA”) (33.6 ml; 0.198 mol). The resulting solution was stirred at 55° C. for 48 h. The reaction was stopped, washed with water and EtOAc. The organic phase was washed several times with saturated NaHCO₃ solution and then with brine. The solvent was removed by distillation to give the desired product as a yellow oil in 100% yield.

### Example 5

### Preparation of Gabapentin Enacarbil and its Derivatives (Step 3 Path B)

AEC-Thiol (R₁ is C₁₂; R₂ is CH(CH₃)₂) (1 eq) was dissolved in THF (4 vol) followed by addition of Gabapentin ester (1 eq) and CF₃CO₂Ag (2 eq). The reaction was stirred at RT and monitored by TLC.

### Example 6

### General Procedure of the Preparation of Gabapentin Enacarbil and its Derivatives

AEC-Thiol (R₁ is C₁₂; R₂ is CH(CH₃)₂) (1 eq) was dissolved in a solvent (20 vol) as listed in Table 1 followed by addition of Gabapentin/Gabapentin tetrabutyl-ammonium salt (1 eq), triethyl amine (optionally) and CF₃CO₂Ag (3 eq). The reaction was stirred at a temperature and for a period of time as listed in Table 1 and was monitored by TLC.

TABLE 1

| | | TEA | | Gabapentin- | Period | Temper- |
|---|---|---|---|---|---|---|
| Entry | Solvent | [1 eq] | Gabapentin | NBu₄ salt | of time | ature |
| 1 | MeOH | − | − | + | 4 days | RT |
| 2 | MeOH | + | − | + | 4 days | RT |
| 3 | THF | − | − | + | 4 days | RT |
| 4 | THF | + | − | + | 4 days | RT |
| 5 | MeCN | − | − | + | 4 days | RT |
| 6 | MeCN | + | − | + | 4 days | RT |
| 7 | MeOH | − | − | + | 3 days | 50° C. |
| 8 | MeOH | + | − | + | 3 days | 50° C. |
| 9 | MeCN | − | − | + | 3 days | 50° C. |
| 10 | MeCN | + | − | + | 3 days | 50° C. |
| 11 | MeOH | − | + | − | 2 days | RT |
| 12 | MeOH | + | + | − | 2 days | RT |
| 13 | H₂O | − | + | − | 2 days | RT |
| 14 | H₂O | + | + | − | 2 days | RT |
| 15 | MeOH | − | + | − | 2 days | 50° C. |
| 16 | MeOH | + | + | − | 2 days | 50° C. |
| 17 | H₂O | − | + | − | 2 days | 50° C. |
| 18 | H₂O | + | + | − | 2 days | 50° C. |

### Example 7

### Preparation of Gabapentin Enacarbil in a One Pot Reaction (Steps 3+4, Path A)

S-ethyl O-1-(isobutyryloxy)ethyl carbonothioate (“AEC-ethyl-thiol”) (1 eq) was dissolved in CH₂Cl₂ (4 vol) and cooled in ice-water bath followed by dropwise addition of freshly distilled SO₂Cl₂ (1 eq). The reaction was stirred at this temperature for 30 min and then for additional 30 min at RT. Then Gabapentin tetrabutyl-ammonium salt (1 eq) was added and the reaction mixture was stirred at RT and monitored by TLC.

### Example 8

### Preparation of GBPE (One-Pot Reaction) (Step 3+4, Path A)

AEC-Et-thiol (0.3 g; 1.36 mmol) was cooled in ice-water bath followed by addition of sulfuryl chloride (0.42 g; 3.26 mmol). The reaction was stirred at 0° C. for 30 min and then at room temperature for additional 1.5 h. Then the obtained mixture was added dropwise to a premixed solution of Gabapentin (0.46 g; 2.72 mmol) and NaOH (0.16 g; 4.08 mmol) in MTBE/H2O is 10:1(15 ml/1.5 ml) and the resulting reaction mixture was stirred at room temperature for over night. The reaction was stopped, washed with water and the phases were separated. The aqueous phase was acidified with 0.1N NaHSO4, extracted with MTBE and the organic phase was evaporated. The above experiment was repeated several times to give GBPE in 40-60% yield.

1. Acid ethyl carbonate-chloride (“AEC-Cl”), having the following formula 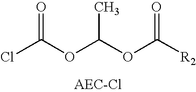
wherein R₂ is H, C₁-C₁₉ alkyl or C₆-C₁₉ aryl.

2. AEC-Cl of claim 1, wherein R₂ is isopropyl.

3. A process for preparing AEC-Cl comprising: combining AEC-thiol having the following formula: 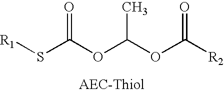
wherein R₁ is C₅-C₂₀ alkyl and R₂ is H, C₁-C₁₉ alkyl or C₆-C₁₉ aryl, with a chlorinating agent.

4. The process of claim 3, wherein the chlorinating agent is selected from a group consisting of: SO₂Cl₂, SOCl₂, POCl₃, PCl₃ and PCl₅.

5. The process of claim 4, wherein the chlorinating agent is SO₂Cl₂.

6. The process of claim 3, wherein the temperature of the AEC-thiol is of about 5° C. to about 0° C. at the time it is combined with the chlorinating agent.

7. The process of claim 3, further comprising adding a solvent.

8. The process of claim 7, wherein the solvent added is an inert polar solvent.

9. The process of claim 8, wherein the solvent is selected from a group consisting of C₅ to C₁₂ hydrocarbons, C₆ to C₁₂ aromatic hydrocarbons, and C₁ to C₂ chloroalkanes.

10. (canceled)

11. A process for preparing a prodrug of GBP comprising: converting AEC-Cl of claim 1 into a prodrug of GBP.

12. The process of claim 11, comprising combining AEC-Cl with a GBP derivative of the following formula: 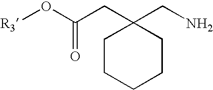
in the presence of a solvent, wherein R₃′ is H, primary ammonium, secondary ammonium, tertiary ammonium, quaternary ammonium or tri-substituted silyl, wherein each individual substituting group of the ammonium or silyl is a hydrogen or a C₁-C₁₀ alkyl, independently of each other.

13. The process of claim 12, wherein AEC-Cl is prepared by combining AEC-thiol having the following formula: 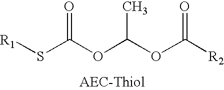
wherein R₁ is C₅-C₂₀ alkyl and R₂ is H, C₁-C₁₉ alkyl or C₆-C₁₉ aryl, with a chlorinating agent.

14. The process of claim 11, wherein the prodrug of GBP is GBPE.

15. A process for preparing GBPE derivative comprising: combining AEC-Cl of claim 1 with a GBP derivative of the following formula: 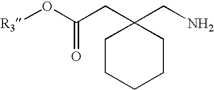
in the presence of a solvent, wherein R₃″ is C₁-C₁₉ alkyl, allyl or benzyl.

16. (canceled)

17. (canceled)

18. (canceled)

19. (canceled)

20. (canceled)

21. (canceled)

22. A one-pot process for preparing a prodrug of GBP comprising: combining AEC-thiol of claim 40 with a chlorinating agent; and adding a GBP derivative of the following formula: 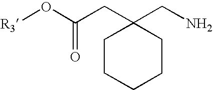
wherein the process is done in the presence of a solvent, wherein R₃′ is H, primary ammonium, secondary ammonium, tertiary ammonium, quaternary ammonium or tri-substituted silyl, wherein each individual substituting group of the ammonium or silyl is a hydrogen or a C₁-C₁₀ alkyl, independently of each other.

23. A one-pot process for preparing GBPE derivative comprising: combining AEC-thiol of claim 40 with a chlorinating agent; and adding a GBP derivative of the following formula: 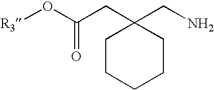
wherein the process is done in the presence of a solvent, wherein R₃″ is C₁-C₁₉ alkyl, allyl or benzyl.

24. (canceled)

25. (canceled)

26. (canceled)

27. A process for preparing a prodrug of GBP comprising: combining the intermediate AEC-Thiol of the following formula: 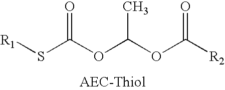
with a GBP derivative of the following formula 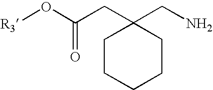
in the presence of an activating agent, wherein R₁ is C₁-C₂₀ alkyl, R₂ is H, C₁-C₁₉ alkyl or C₆-C₁₉ aryl, and R₃′ is H, primary ammonium, secondary ammonium, tertiary ammonium, quaternary ammonium or tri-substituted silyl, wherein each individual substituting group of the ammonium or silyl is a hydrogen or a C₁-C₁₀ alkyl, independently of each other.

28. (canceled)

29. A process for preparing a GBPE derivative comprising: combining the intermediate AEC-Thiol of the following formula: 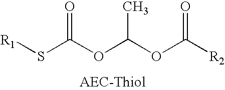
with a GBP derivative of the following formula 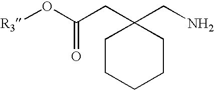
in the presence of an activating agent, when R₁ is C₁-C₂₀ alkyl, R₂ is H, C₁-C₁₉ alkyl, or C₆-C₁₉ aryl, and R₃″ is C₁-C₁₉ alkyl, allyl or benzyl.

30. The process of claim 27, wherein prior to the combining step, AEC-thiol is dissolved in a solvent selected from a group consisting of toluene, tetrahydrofuran (“THF”), MeCN, CH₂Cl₂, and H₂O.

31. The process of claim 30, wherein the solvent is CH₂Cl₂.

32. The process of claim 30, wherein the obtained solution is combined with a GBP derivative in the presence of an activating agent.

33. The process of claim 32, wherein the activating agent is selected from a group consisting of CF₃CO₂Ag, (CF₃CO₂)₂Hg, CF₃SO₃Ag, and (CF₃SO₃)₂Hg.

34. The process of claim 27, further comprising adding a base or an excess amount of GBP derivative.

35. The process of claim 34, wherein the base is an inorganic base or a tertiary amine.

36. (canceled)

37. Chloro ethyl carbonate-thiol (“CEC-thiol”) having the following formula: 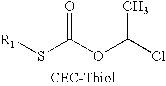
wherein R₁ is C₅-C₂₀ alkyl.

38. (canceled)

39. (canceled)

40. Acid ethyl carbonate-thiol (“AEC-thiol”) having the following formula: 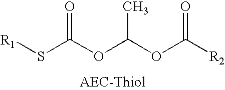
wherein R₁ is C₅-C₂₀ alkyl and R₂ is H, C₁-C₁₉ alkyl or C₆-C₁₉ aryl.

41. (canceled)

42. (canceled)

43. (canceled)

44. A process for the preparation of CEC-Thiol having the following formula: 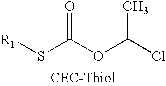
comprising: combining chloroethyl chloroformate (“CEC”) with R₄CH₂SH having the following structure: 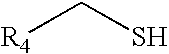
or a salt thereof, wherein R₁ is CH₂R₄ and R₄ is C₄-C₁₉ alkyl.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A process for the preparation of AEC-Thiol having the following formula: 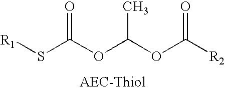
comprising: combining CEC-Thiol of the following formula: 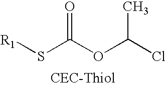
with a carboxylic acid having the formula R₂CO₂H, or its salt, wherein R₁ is C₅-C₂₀ alkyl, and R₂ is H, C₁-C₁₉ alkyl or C₆-C₁₉ aryl.

64. (canceled)

65. (canceled)

66. (canceled)

67. (canceled)

68. (canceled)

69. (canceled)

70. (canceled)

71. (canceled)

72. (canceled)
